(19) United States
(12) Patent Application Publication
(10) Pub. No.: US 2003/0215435 A1
(43) Pub. Date: Nov. 20, 2003

(54) Expression system for effeiciently producing clinically effective lysosomal enzymes (glucocerebrosidase)

(76) Inventor: Susan L Berent, Dallas, TX (US)

Correspondence Address: Eugenia S Hansen, Sidley Austin Brown & Wood, 717 Hardwood Suite 3400, Dallas, TX, 75201, US

(21) Appl. No.: 10/240,687

(22) PCT Filed: Apr. 6, 2001
(86) PCT No.: PCT/US01/11144

(30) Foreign Application Priority Data
Apr. 6, 2000 (US) 60195598

(51) Int. Cl. A61K 38/46; C07H 21/04; C12N 9/24; C12P 21/02; C12N 5/06

(52) U.S. Cl. 424/94.62; 435/200; 435/348; 435/69.1; 435/320.1; 536/23.2

(57) ABSTRACT

The invention as described herein relates to the efficient production of recombinant, clinically effective lysosomal enzymes using a transformed insect cell expression system. For example, to create the expression system of the invention, any insect cell can be transfected with a plasmid comprised of a gene encoding the human glucocerebrosidase gene and genetic elements that enhance its expression. The insect cell transfected with the plasmid encoding glucocerebrosidase secretes synthesized glucocere-brosidase into its growth media. The recombinantly produced clinically effective glucocerebrosidase produced by the insect cell expression system can be used to treat Gaucher's disease.

### TECHNICAL FIELD OF THE INVENTION

The present invention relates to a system for efficiently producing clinically effective glucocerebrosidase.

### BACKGROUND OF THE INVENTION

More than thirty genetically inherited lysosomal storage diseases have been characterized in humans. Lysosomal storage diseases, although relatively rare, can be fatal if left untreated. Ubiquitous among animal cells, lysosomes are intracellular organelles that contain hydrolytic enzymes. Lysosomal storage diseases are caused by the accumulation of a deficient enzyme's substrate in lysosomes, thereby increasing the size and number of lysosomes. An increase in the number and size of lysosomes results in gross pathology specific to the lysosomal storage disease. Examples of lysosomal storage diseases include the following: Fabry disease, caused by a deficiency of α-galactosidase; Farber disease, caused by a deficiency of ceramidase; Gₘ₁ gangliosidosis, caused by a deficiency of β-galactosidase; Tay-Sachs disease, caused by a deficiency of β-hexosaminidase; Niemann-Pick disease, caused by a deficiency of sphingomyelinase; Schindler disease, caused by a deficiency of α-N-acetylgalactosaminidase; Hunter syndrome, caused by a deficiency of iduronate-2-sulfatase; Sly syndrome, caused by a deficiency of β-glucuronidase; Hurler and Hurler/Scheie syndromes, caused by a deficiency of iduronidase; I-Cell/San Fillipo syndrome, caused by a deficiency of mannose 6-phosphate transporter; and Gaucher disease, caused by a deficiency of human glucocerebrosidase (GC).

Gaucher disease is the most common lysosomal storage disease in the human population and is discussed herein as merely an example of the need to provide replacement therapy of appropriate lysosomal enzyme to the corresponding lysosomal enzyme deficiency disease.

Gaucher disease is caused by a deficiency of GC activity, which hydrolyzes the β-glycosidic linkage between the ceramide and glucose moieties of glucocerebroside. This glycolipid cannot be catabolyzed at a sufficient rate in patients with Gaucher disease due to the decreased enzymatic activity and thus, accumulates in reticuloendothelial cells of the bone marrow, spleen, and liver. Complications of Gaucher disease include bone marrow expansion, bone deterioration, hypersplenism, hepatomegaly, thrombocytopenia, anemia, and lung disorders. Three clinical forms of Gaucher disease have been described to date: type I (adult, non- neuronopathic), type 2 (infantile, acute neuronopathic), and type 3 (juvenile, subacute neuronopathic). Gaucher disease, an autosomal recessive disease, is most prevalent in the Ashkenazi Jewish population, where one in eighteen is a carrier. Over five thousand people in the United States alone are afflicted with this disease, 99% of whom are considered to have the type 1 clinical form.

The current treatment for Gaucher disease involves the replacement of the deficient GC with active GC, made possible with the knowledge of the GC sequence and recombinant DNA technology (Tsuji et al., 1986; Sorge et al., 1985; Sorge et al., 1986). Administering exogenous GC, termed enzyme replacement therapy, has significantly improved the lives of many Gaucher patients. Enzyme replacement therapy reduces the symptomatic effects of Gaucher disease and reverses the hepatic, splenic, and hematologic manifestations of the disease (Pastores et al., 1993).

Unfortunately, the benefits from enzyme replacement therapy are costly. At this time, there are only two methods for commercially producing clinically effective, purified human GC. The first method involves purifying GC from pooled human placentae, currently produced by Genzyme Corporation as Ceredase™. Approximately 500 to 2000 kilograms of placenta (equivalent to 2,000-8,000 placentae) are required to treat each Gaucher disease patient every two weeks. (Radin, U.S. Pat. No. 5,929,304).

Interestingly, placental GC does not possess optimal pharmacokinetic properties for treating Gaucher disease. Because glycoproteins are cleared from the circulation and differentially taken up by various cell types through plasma membrane receptors, producing GC with N-glycan terminal sugars that favor uptake into the target cells results in a more effective distribution of GC. Deposits of glucocerebroside in Gaucher patients are found in non-parenchymal cells, such as Kupffer cells and macrophages, but not in parenchymal cells, such as hepatocytes. The non-parenchymal cells do not preferentially take up native placental GC. The existence of ubiquitous mannose-recognizing receptors on macrophage membranes (Kawasaki et al., 1978; Baynes and Wold 1976; Stahl et al., 1978) suggested that GC with at least one exposed mannose residue could be differentially targeted to phagocytic cells to achieve therapeutic effect. Sequential removal of NeuAc, Gal, and GlcNAc residues from the native human placental GC by the enzymatic activity of neuraminidase, galactosidase, and N-acetylglucosaminidase (Furbish et al., 1981) generated modified GC with terminal mannose residues that bad a significantly increased rate of clearance from the circulation and increased specific uptake by Kupffer cells. The increased effectiveness of the remodeled GC is believed to be due to the exposure of terminal mannose residues, the removal of sialic acid residues (which decrease the rate of clearance of glycoproteins), and the removal of N-glycan terminal galactose residues (which preferentially direct glycoproteins to cells containing galactose receptors, such as hepatocytes) (Ashwell and Morell, 1974). As a result of this research, the commerical preparation of clinically effective GC involves remodeling of native GC as described by Furbish et al., 1981.

A second method of commercially producing GC, which eliminates the safety concerns associated with GC isolated from human tissue, involves in vitro cell culture. Genzyme Corporation produces Cerezyme™ from Chinese hamster ovary cells transformed with a plasmid encoding a human GC DNA sequence (Rasmussen et al., U.S. Pat. No. 5,236,838, hereinafter the '838 patent). The carbohydrate chains of GC produced in this system must be remodeled in the same way that the placental GC is remodeled to render the final GC product clinically effective.

Carbohydrate remodeling of GC into its clinically effective form is a time-consuming and expensive process. This process requires sequential application of three enzymes to create N-glycans with terminal mannose residues that convert the placental GC or the CHO-synthesized GC into its clinically effective form. Both methods are expensive: the approximate cost of treating a 50 kilogram patient with Gaucher disease is $70,000 to $300,000 per year (Radin, supra). Currently, there is no commercially employed method to produce clinically effective GC in animal cells without the time-consuming and expensive process of carbohydrate remodeling. Because of its crucial role in determining GC clinical efficacy, it is worthwhile to consider the process of human protein glycosylation.

To better understand the carbohydrate remodeling process, a brief overview of human glycosylation is presented herein. Proteins synthesized in mammalian cells destined for secretion or transport to the Golgi, lysosomes, or plasma membrane, are covalently modified with carbohydrates in the endoplasmic reticulum and Golgi apparatus. The addition of such carbohydrates to proteins facilitate in vivo functionality by directing localization of the mature glycoprotein and, in some cases, inducing correct protein conformation. The process of protein glycosylation begins with the transfer of a preformed oligosaccharide containing 14 sugar residues comprised of N-acetylglucosamine, mannose, and glucose from dolichol to specific asparagine residues of the protein. Next, glycosidases may remove glucose and mannose residues in the endoplasmic reticulum. In the Golgi apparatus, the protein may be left unmodified, leaving N-glycans described as the “high mannose,” type or the protein may be further processed by the addition of more sugars, resulting in N-glycans described as “complex” oligosaccharides. Complex oligosaccharides include sialic acid, fucose, galactose, mannose 6-phosphate, and N-acetylglucosamine residues.

The process of endogenous GC glycosylation follows the same pattern as other lysosomal enzymes. The glycosylation of human placental GC results in a mature GC with an apparent molecular mass of 66 kDa due to glycosylation at four of five consensus sequences for asparagine-linked glycosylation. One of these sites must be glycosylated to confer enzymatic activity, thus requiring GC production in a eukaryotic system. Approximately 25% of the N-glycans of placental GC are of the high-mannose type with the remaining N-glycans being the complex type.

Despite the difficulty of producing clinically effective GC, there are examples of recombinant GC being produced in heterologous systems. For example, the baculovirus expression system can be harnessed to produce GC in virally infected insect cell lines (Ginns et al., U.S. Pat. No. 6,074,864, hereinafter the '864 patent). Ginns reported that this expression system yielded 2.2 mg of GC per liter. The majority of the GC produced by this system and in the baculovirus system studied by Grabowski et al., (1989) was found to be cell associated. In addition to the very low yield of GC produced by the method of the '864 patent, numerous disadvantages to the expression system of the '864 patent exist. First, because the host insect cells are killed at the end of each infection cycle, GC expression is only transient, requiring constant infections of new cells in order to continuously produce GC. In order to constantly infect new cells, large stocks of two cell lines must be cultured: one cell line must be maintained to infect and express GC, and the second cell line must be maintained to generate virus. Second, the biological authenticity of expressed protein is not guaranteed, because the cell machinery necessary for post-translational modifications in insect cells is inactivated in the late stages of infection. This leads to an increase in the heterogeneity of the expressed GC and an apparent molecular mass ranging from 52 kDa to 67 kDa ('864 patent). For this reason, the amount of clinically effective GC produced by the '864 patent may actually be lower than the yield reported. Third, the purification of recombinant GC from virally-infected insect cells is inconvenient, requiring detergent-mediated extraction and a complex purification scheme. The low GC yield, inefficient GC secretion, and complicated purification scheme are all major disadvantages of the baculovirus-expression system claimed in the '864 patent.

The '838 patent claims a CHO-expression system comprising a recombinant GC at least 95% identical to an amino acid sequence of primate GC. The expression system described in the '838 patent discloses both baculovirus-infected insect cells and transfected mammalian cells (CHO cells) to express recombinant GC. According to the '838 patent, one to ten milligrams of recombinant GC per liter of CHO cells was recovered. In the CHO expression system, the recombinant GC was detected intracellularly after extraction with detergent and in the growth media, indicating that only a portion of the GC was secreted into the growth media. The GC harvested from within the cells had a lower molecular weight and was sensitive to endoglucosaminidase H and endoglucosaminidase F, indicating the intracellular GC was of the high mannose type. Conversely, the secreted GC was resistant to endoglucosaminidase H. Cerezyme™, which is produced by this method, differs from placental GC by the presence of a histidine in place of arginine at position 495 of the mature GC and by the absence of any high mannose type N-glycans.

Recombinant GC, produced by the method of the '838 patent, also requires remodeling as described by Furbish et al., (1981) to be clinically effective. The remodeled recombinant GC and placental GC were found to have different cell type distributions in vivo with approximately twice as much recombinant GC reaching the targeted Kupffer cells (Friedman, U.S. Pat. No. 5,549,892, hereinafter the '892 patent). The increased clinical efficacy of the recombinant GC was attributed to either the small difference in the amino acid sequence or to differences in carbohydrate composition. The carbohydrate structure of the CHO-expressed GC has a greater number of fucose and N-acetylglucosamine residues than the remodeled placental GC.

In the Radin patent (U.S. Pat. No. 5,929,304, hereinafter the '304 patent), an expression system to produce GC and α-L-iuronidase in transgenic tobacco plants is claimed. In the method of the '304 patent, the GC is expressed as its naturally occurring sequence or with an antibody-specific epitope. The purification of GC from the plant requires harvesting the plant and extracting the membrane-associated GC in a laborious and potentially costly process involving detergent extraction. Not only is the process for GC purification labor-intensive, but also as another disadvantage of this system, differences exist between the glycosylation patterns in plants and humans that will affect the clinical efficacy of plant-expressed GC (Doran, 2000). Unlike humans, plant glycoproteins do not contain α1,6-fucose residues linked to the innermost N-acetylglucosamine residue. In vivo, α1,6-fucosylation protects N-glycans against hydrolysis by glycoasparaginase (Noronkoski et al., 1997), thus, protecting the mannose terminated N-glycans that are important for GC's clinical efficacy. In addition, plant N-glycans contain a plant specific β1,2-xylose residue attached to the β-linked mannose residue of the core N-glycan (Staudacher et al., 1999), which is known to be highly immunogenic and may be allergenic (van Ree et al., 2000). Thus, plants have not been considered appropriate candidates for the expression of therapeutic lysosomal enzymes due to their glycosylation profile (Altmann, 1997; Bakker et al., 2001). The apparent increase in molecular mass of plant-produced GC was reported in the '304 patent to be 8 kDa, in contrast to Cerezyme's™ molecular mass increase of 4.8 kDa, indicating yet another difference between Cerezyme™ and plant-produced GC having unknown consequences.

Studies have shown that most of the insect cell lines used for recombinant protein expression are unable to perform complex glycosylation due to the lack of, or limited expression of galactosyltrasferases and sialyltransferases, enzymes that are involved in complex N-glycan synthesis (Takahashi et al., 1999; Hollister and Jarvis, 2001). Therefore, endogenous insect cell proteins contain little or no complex types of N-glycans (Kubelka et al., 1994). Also, when the baculovirus-insect cell expression system is used for producing recombinant glycoproteins, little or no detectable N-glycans containing terminal sialic acid are found (Kulakosky et al., 1998). The inability of insect cells to perform complex glycosylation, which has been considered a limitation for expressing glycoproteins in this system (Altmann, 1997), is actually an advantage for expressing lysosomal enzymes intended for therapeutic purposes. Because insect-produced proteins predominantly contain paucimannose type N-glycans (Kulakosky et al., 1998; Takahashi et al., 1999), with at least one non-reduced terminal mannose residue, the insect cell expression system eliminates the need for the time-consuming and costly enzymatic remodeling steps that are required to produce clinically effective GC isolated from other eukaryotic cells, such as CHO cells.

In the Iatrou patent (U.S. Pat. No. 5,759,809, hereinafter the '809 patent), a method for enhancing heterologous protein production in insect cells is claimed. In order to increase heterologous protein production, the insect cells are transfected with a plasmid encoding, in addition to the protein desired, genetic elements including an insect cell promoter and a baculovirus enhancer. The plasmid may also encode the baculoviral IE-1 gene product, a general transcriptional regulator. Either through infection or transfection, the expression cassette can direct insect cells to synthesize the desired protein in large quantities. This patent is incorporated herein by reference. However, the '809 pateent does not teach the production of clinically effective lysosomal enzymes.

A need exists for a simple expression system that can provide an abundant supply of GC in its clinically effective form without the complication and cost associated with carbohydrate chain remodeling. Recombinant GC can be produced at low levels using several genetically engineered organisms, but clinically effective GC must contain N-glycans with terminal mannose sugars, requiring expression in a eukaryotic organism. Because the majority of heterologous GC produced in eukaryotic organisms is membrane-associated, a complex purification is required to prepare GC from the cells. A much better approach for efficiently producing clinically effective GC in a heterologous expression system is to engineer an expression vector for a system that will produce high levels of mature GC in a soluble and clinically effective form.

The heterologous expression system described herein secretes mature, clinically effective GC at a high yield without the need for carbohydrate remodeling. Therefore, the main advantages of the expression system described herein for GC are (1) the expression of GC in a stably transformed expression system; (2) a consistently higher level of GC expression than in baculovirus or mammalian cell expression systems; (3) production of GC in a soluble form secreted to the media; and (4) proper glycosylation modifications for GC, requiring no enzymatic carbohydrate remodeling to be clinically effective. This expression system described and claimed herein provides for a more effective, economical, and simpler approach to manufacturing recombinant GC.

### BRIEF DESCRIPTION OF THE DRAWINGS

FIG. 1 is a flow diagram of expression vector pIE1/153A.GC-B construction. The plasmid labeled pBLSKm is the pBluescript®SK(-) plasmid (Stratagene, Genbank Accession No. X52324). The crosshatched regions on the depicted vectors denoted “Amp^{R}” encode a gene that confers ampicillin resistance. The lightly dappled regions on the depicted vectors denoted “ColE1 origin” and “f1 origin” are replication origins recognized by Escherichia coli. Plasmid pBLSKm-GC1a containing human GC cDNA sequences depicted as the plain white region encodes human GC (Tsuji et al., 1986) and was obtained as the I.M.A.G.E. Consortium CloneID 512548 from the TIGR/ATCC Special Collection of human cDNA clones (Lennon et al.,1996). Plasmid pBLSKm-GC1a was sequenced to ensure that the coding region for GC corresponded to the published GC sequence (Tsuji et al., 1986). The plain white region denoted “GC1a (BamHI, HindIII)” encodes human GC as exemplified from nucleotide 94 to nucleotide 1492 of SEQ ID NO: 1. The plain white region denoted PCRGCwt2 (BamHI, SphI) is from pBLSKm-PCRGCwt2 (FIG. 2), contains the sequence as exemplified in SEQ ID NO:1 from nucleotide 1489 to nucleotide 1571, encodes human GC as exemplified in SEQ ID NO:2 from amino acid 493 to amino acid 516 and was ligated to the BamHI end of the “GC1a (BamHI, HindIII)” fragment to form the C-terminal end of GC. Oligonucleotides having the sequences of SEQ ID NO:8, SEQ ID NO:9, SEQ ID NO:10, and SEQ ID NO:11 were annealed and ligated together to encode the GC secretion signal and eight amino acid residues of the mature GC N-terminus as exemplified in SEQ ID NO:12. The resulting fragment was ligated to the HindIII end of the “GC1a (BamHI, HindIII)” fragment to form the amino-terminal end of GC. The plain white region denoted “GC” encodes human GC protein as exemplified by SEQ ID NO:2. The expression vector pIE1/153A contains a pBluescript® SK(+) backbone (Genebank Accession No. X52325), denoted as the pBLSKp backbone, and elements for high expression (Lu et al., 1997). The dotted regions on the depicted vectors denoted “actin” and “actin promoter” are the actin gene and actin gene promoter from the Bombyx mori genome. The single hatched regions on the depicted vectors denoted “HR3 element” is the 1.2 kB enhancer from the Bombyx mori NPV genome. The darkened regions on the depicted vectors denoted “IE1 gene” is the immediate early gene from the Bombyx mori genome.

FIG. 2 is a flow diagram of the construction of two different vectors using PCR: pBLSKm-PCRGCwt2 and pBLSKm-PCRGCsr2, both encoding human GC. Plasmid pBLSKm-GC1a containing human GC cDNA sequences depicted as the plain white region and labeled “GC cDNA” encodes human GC (Tsuji et al., 1986) and was obtained as the I.M.A.G.E. Consortium CloneID 512548 from the TIGR/ATCC Special Collection of human cDNA clones (Lennon et al., 1996). Plasmid pBLSKm-GC1a was sequenced to ensure that the coding region for GC corresponded to the published GC sequence (Tsuji et al., 1986). The plasmid labeled pBLSKm is the pBluescript®SK(−) plasmid from Stratagene (Genbank Accession No. X52324). Plasmid pBLSKm-GC1a and primers SEQ ID NO:5, SEQ ID NO:6, and SEQ ID NO:7 were used during PCR to remove 3′ non-coding sequences and to add cloning sites. The sequences of the fragments generated by PCR were confirmed to be correct by nucleotide sequencing. The sequence of the BamHI, SphI fragment from pBLSKm-PCRGCwt2 encoding the 24 C-terminal amino acid residues of human GC reported by Tsuji et al. (1986) can be found in SEQ ID NO:1 from nucleotide 1489 to nucleotide 1571. The sequence of the BamHI, SphI fragment from pBLSKm-PCRGCsr2 encoding the 24 C-terminal amino acid residues of human GC reported by Sorge et al. (1985; 1986; Genbank Accession No. M16328) can be found in SEQ ID NO:3 from nucleotide 1489 to nucleotide 1571. The crosshatched regions on the depicted vectors denoted “Amp^{R}” encode a gene that confers ampicillin resistance. The lightly dappled regions on the depicted vectors denoted “ColE1 origin” and “f1 origin” are replication origins recognized by E. coli.

FIG. 3 is a comparison of the carboxy-terminal ends of two sequences for human glucocerebrosidase. Note the difference in amino acid position 514, which is arginine in SEQ ID NO:2 and histidine in SEQ ID NO:4, encoded by SEQ ID NO:1 and SEQ ID NO:3, respectively.

FIG. 4 is a bar graph demonstrating that both expression vectors encoding SEQ ID NO:1 and SEQ ID NO:3 direct enzymatically active GC production and secretion in all three cell lines, Bm5, High Five™, and Sf21. Bm5, High Five™, and Sf21 cells were transfected with pIE1/153A.GC-B (containing SEQ ID NO:1), pIE1/153A.GC-C (containing SEQ ID NO:3), and pIE1/153A (the vector without insert). Media aliquots from each cell population were tested for β-glucosidase activity. The asterisk indicates that the Bm5 cell population containing the vector without insert expressed essentially zero GC activity.

FIG. 5 is a Western blot comparing the molecular mass of GC secreted (lanes 5-7) into serum-free media or maintained intracellularly (lanes 11-13) to that of Cerezyme™. High Five™ cells were transfected with the pIE1/153A.GC-B containing SEQ ID NO:1 (lanes A), pIE1/153A.GC-C containing SEQ ID NO:3 (lanes B), and the vector without insert, pIE1/153A (lanes C). After growing for three days in serum-free media, 10.0 82 L samples of media and extracts from 25,000 cells were resolved on a 9% SDS-PAGE gel, transferred by Western blotting, and probed with the GC-specific antibody NN1274.

FIG. 6 is a graph of GC activity secreted by three cell lines, each produced by single cell clones transformed with the GC-encoding plasmid pIE1/153A.GC-B. At each time period designated, aliquots of media were tested for GC activity.

FIG. 7 is a Coomassie® Blue stained SDS-PAGE gel of media aliquots from culture supernatants of Bm5, High Five™, and Sf21 cells either untransformed (denoted by “−”), or transformed (denoted by “+”) with the GC-encoding plasmid pIE1/153A.GC-B. 4.0 ™L of media aliquots were taken at the days indicated on the figure from Bm5 and Sf21 culture supernatants whereas 2.0 μL was taken from High Five™ culture supernatants and loaded on the gel. The amounts of Cerezyme™ indicated on the figure were loaded for comparision. The samples were resolved on a 9% SDS-PAGE gel.

FIG. 8 is a Western blot of media aliquots from culture supernatants of Bm5, High Five™, and Sf21 cells either untransformed (denoted by “−”), or transformed (denoted by “+”) with the GC-encoding plasmid pIE1/153A.GC-B. 2.0 μL of media aliqouts were taken at the days indicated on the figure from Bm5and Sf21 culture supernatants whereas 1.0 μL was taken from High Five™ culture supernatants and loaded on the gel. The amounts of Cerezyme™ indicated on the figure were loaded for comparision. The samples were resolved on a 9% SDS-PAGE gel, transferred by Western blotting, and probed with the GC-specific antibody NN1274.

### SUMMARY OF INVENTION

One aspect of the invention is a pharmaceutical composition comprising clinically effective GC produced by an insect expression system, wherein the insect cells are transformed with a vector encoding GC. The vector that encodes GC may contain SEQ ID NO:1 or SEQ ID NO:3. The vector may optionally encode a secretion signal, as exemplified by amino acids 1-19 of SEQ ID NO:12. Additionally, the vector may include a promoter sequence and an enhancer sequence functionally linked to the expression of GC. An exemplary promoter region is the actin gene promoter from the Bombyx mori genome. An exemplary enhancer region is the 1.2 kB enhancer from the Bombyx mori NPV genome. The vector may also encode a general transcriptional regulator, such as the IE-1 gene from the Bombyx mori genome. Insect cells that may be part of the expression system include those from the species of Bombyx mori, Spodoptera frugiperda, or Trichoplusia ni. The clinically effective GC produced by the insect expression system possesses asparagine-linked terminal mannose residues.

Yet another aspect of the invention is a method for treating individuals with deficiencies in GC, wherein the method includes introducing into these individuals clinically effective recombinant GC produced by insect cells.

A further aspect of the invention is an expression system that is comprised of an insect cell transformed with a vector encoding GC that produces clinically effective GC. The vector that encodes GC may contain SEQ ID NO:1 or SEQ ID NO: 3. The vector may optionally encode a secretion signal, as exemplified by amino acids 1-19 of SEQ ID NO: 12. Additionally, the vector may include a promoter sequence and an enhancer sequence functionally linked to the expression of GC. An exemplary promoter region is the actin gene promoter from the Bombyx mori genome. An exemplary enhancer region is the 1.2 kB enhancer from the Bombyx mori NPV genome. The vector may also encode a general transcriptional regulator, such as the IE-1 gene from the Bombyx mori genome. Insect cells that may be part of the expression system include those from the species of Bombyx mori, Spodoptera frugiperda, or Trichoplusia ni. The clinically effective GC produced by the insect expression system possesses asparagine-linked terminal mannose residues.

Yet another aspect of the invention is a method of producing clinically effective GC comprising the steps of developing a vector that encodes GC, introducing the developed vector into at least one cell that is capable of receiving the vector and as acting as host to the vector, nurturing the insect cell that contains the vector so the GC is transcribed and translated in its clinically effective form, and recovering the insect cell-produced GC. The vector that encodes GC may contain SEQ ID NO:1 or SEQ ID NO:3. The vector may optionally encode a secretion signal, as exemplified by amino acids 1-19 of SEQ ID NO: 12. Additionally, the vector may include a promoter sequence and an ehancer sequence functionally linked to the expression of GC. An exemplary promoter region is the actin gene promoter from the Bombyx mori genome. An exemplary enhancer region is the 1.2 kB enhancer from the Bombyx mori NPV genome. The vector may also encode a general transcriptional regulator, such as the IE-1 gene from the Bombyx mori genome. Insect cells that may be part of the expression system include those from the species of Bombyx mori, Spodoptera frugiperda, or Trichoplusia ni. The clinically effective GC produced by the insect expression system possesses asparagine-linked terminal mannose residues.

Furthermore, another aspect of the invention is a method of producing clinically effective GC comprising the steps of creating a vector that encodes GC with a signal sequence for secretion functionally linked to an enhancer and a promoter, wherein the vector also encodes a structural gene that enhances transcription as well as a structual gene that is a detectable marker, introducing the created vector into an insect cell, growing the insect cell, synthesizing and secreting clinically effective GC under conditions favorable for growth and replication, and collecting the secreted, recombinantly synthesized, and clinically effective GC. The vector that encodes GC may contain SEQ ID NO:1 or SEQ ID NO:3. The vector may optionally encode a secretion signal, as exemplified by amino acids 1-19 of SEQ ID NO: 12. Additionally, the vector may include a promoter sequence and an ehancer sequence functionally linked to the expression of GC. An exemplary promoter region is the actin gene promoter from the Bombyx mori genome. An exemplary enhancer region is the 1.2 kB enhancer from the Bombyx mori NPV genome. The vector may also encode a general transcriptional regulator, such as the IE-1 gene from the Bombyx mori genome. Insect cells that may be part of the expression system include those from the species of Bombyx mori, Spodoptera frugiperda, or Trichoplusia ni. The clinically effective GC produced by the insect expression system possesses asparagine-linked terminal mannose residues.

### DETAILED DESCRIPTION OF THE PREFERRED EMBODIMENTS

This section provides a general discussion of preferred methodologies to develop preferred transfected cells and vectors, which includes, but is not limited to, the preferred components of expression cassettes containing lysosomal enzymes (for example, GC), and the overall process of producing clinically effective lysosomal enzymes. The methodolgies are merely presented to enable those skilled in the art of molecular biology to reproduce the claimed invention. Other methodologies can be used as known by those skilled in the art, as long as the resulting expression system produces a clinically effective lysosomal enzyme (for example, GC) in a sufficient quantity to be applied pharmaceutically.

The present invention relates to a heterologous expression system capable of expressing a lysosomal enzyme that is clinically effective in a significant quantity. The expression system is comprised of a transfected insect cell, wherein the insect cell is transfected with a vector containing an expression cassette encoding a human lysosomal enzyme. The expression cassette of the transfection vector has, in addition to a coding sequence of a human lysosomal enzyme, genetic elements to support a high level of expression. Genetic elements, nucleotide sequences, may initiate transcription, increase transcription, or encode peptides for localization. The transfection vector used to create the expression system of the current invention also encodes a detectable marker to differentiate transfected insect cells from non-transfected insect cells. The expression system herein described results in the secretion of clinically effective human lysosomal enzyme secretion into the insect cell's extracellular environment. The expression system of the current invention is capable of producing a clinically effective lysosomal enzyme at unprecedented levels, making the process highly efficient.

More specifically, the invention relates to expression cassettes containing promoters and enhancers identified from insects, a recombinant expression cassette containing a DNA sequence representing a lysosomal enzyme gene functionally linked to an insect cellular promoter, transplacement fragments containing recombinant expression cassettes, vectors having transplacement fragments, and enhancer components and stable lines of various insect cells. To provide for a better understanding of the invention, certain definitions are provided as follows:

An “expression system” is defined specifically herein as a heterologous expression system that includes an insect cell containing the elements of the vector encoding a lysosomal enzyme, already defined above. The expression system results in the secretion of a clinically effective lysosomal enzyme into the insect cell's extracellular environment. The expression system of the current invention is capable of producing clinically effective lysosomal enzyme at unprecedented levels, making the process highly efficient.

A “vector” is defined herein as a nucleic acid composition that includes the expression cassette and DNA sequences that provide for replication and selection preferably in bacteria (e.g. E. coli) for amplification. In addition to the expression cassette, the vector may also encode for other gene products. The vector may be a plasmid.

An “expression cassette” is defined herein as a nucleotide sequence encoding from its 5′ to 3′ direction: (1) a promoter sequence; (2) a signal sequence for secretion; and 3) a nucleotide coding sequence for a lysosomal enzyme. A preferred sequence is GC. The expression may optionally include an enhancer. The sequences for all of the elements are functionally linked to one another. The expression cassette is capable of directing the expression and secretion of a lysosomal enzyme in its active form. Additionally, and as known to those in the art, the expression system can include additional nucleic acid sequences for terminating transcription and additional nucleic acid sequences for initiating and terminating translation.

The “promoter” is defined herein as a DNA sequence that initiates and directs the transcription of a heterologous gene into an RNA transcript in cells. The promoter can be any DNA sequence that initiates and directs transcription. For example, the promoter may be a mammalian promoter such as the cytomegalovirus immediate early promoter, the SV40 large T antigen promoter, or the Rous Sarcoma virus (RSV) LTR promoter. Alternatively, the promoter may be derived from an insect cell, such as the actin gene promoter from Bombyx mori, the ribosomal gene promoter, the histone gene promoter, or the tubulin gene promoter.

A “signal sequence” is defined herein as a nucleotide sequence that encodes an amino acid sequence that initiates transport of a protein across the membrane of the endoplasmic reticulum. Additionally, signal sequences could initiate peptide secretion. A signal sequence localizes a synthesized protein. Although other signal sequences could be used, an amino acid sequence of an exemplary signal sequence for GC is given by amino acid residues 1-19 of SEQ ID NO:12.

An “enhancer” is defined herein as any nucleic acid that increases transcription when functionally linked to a promoter regardless of relative position (for example, a cis-acting enhancer). An exemplary enhancer for GC expression a 1.2 kB BmNPV enhancer region defined in the '809 patent. “Functionally linked” is defined herein as the influential relationship between two or more nucleotide regions. For example, the actin gene promoter is functionally linked to a lysosomal enzyme gene if it controls the transcription of the gene and it is located on the same nucleic acid fragment as the gene. In another example, an enhancer is functionally linked to a lysosomal enzyme gene if it enhances the transcription of that gene and it is located on the same nucleic acid fragment as the gene.

Other protein products that could be encoded by the vector include detectable markers and transcription regulators. “Detectable markers” are defined herein as genes that allow for the detection of cells that contain the elements of the vector defined above over cells which do not. Detectable markers include reporter genes and selection genes. A reporter gene encodes a foreign protein not required for cell survival. Suitable reporter genes include the gene encoding for green fluorescent protein and the β-galactosidase gene. Like reporter genes, a selection gene encodes a foreign protein required for cells to live under certain conditions. As an example, selection genes encode antibiotic resistance. Other gene products that could be encoded by the vector may confer functionality. For example, the IE-1 protein of nuclear polyhedrosis viruses (Huybrechts et al., 1992 or Genbank Accession No. X58442) or the herpes simplex virus VP 16 transcriptional activator are proteins that may be included on the vector to promote the expression level.

“Secrete” or “secretion” is defined herein as the active export of lysosomal enzyme from a host cell into the extracellular environment. Secretion occurs through a secretory pathway in the host cell. For example, in eukaryotic host cells, secretion involves the endoplasmic reticulum and Golgi apparatus cellular components.

“Transcription” is defined herein as the biosynthesis of an RNA molecule from a DNA template strand. The sequence of the synthesized RNA molecule is complementary to the sequence of the DNA template strand.

“Transfection” as defined herein refers to a technique for introducing purified nucleic acid into cells by any number of methods known to those skilled in the art. These methods include, but are not limited to, electroporation, calcium phosphate precipitation, cationic lipids, DEAE dextran, liposomes, receptor-mediated endocytosis, particle delivery, and injection. Cells can be transfected using an appropriate introduction technique known to those in the art (e.g., liposomes). In a preferred embodiment of the invention described herein, the vector is introduced into the insect cells by mixing the DNA solution with Lipofectin™ (GIBCO BRL) and adding the mixture to the cells.

“Transformation” as defined herein refers to the insertion of introduced DNA into the genome of the organism in which the DNA was introduced.

“Translation” is defined herein as the linking of amino acids carried by transfer RNA molecules in an order specified by the order of the codons along a messenger RNA molecule. The product of translation is a protein.

“Insect cells” is defined herein as any living insect cell of any species. In a preferred embodiment of the invention described herein, the insect cells from the species Bombyx mori, Spodopterafrugiperda, and Trichoplusia ni were used. Although the use of insect cells is preferred, it is to be understood that any cell line able to express and secrete lysosomal enzymes (for example, GC) in their clinically effective forms can be used.

“Clinically effective” as defined herein describes lysosomal enzymes that function as well or better than native lysosomal proteins in patients deficient in the endogenous lysomal enzyme.

### GENERAL PREPARATIONS AND METHODOLOGIES

General DNA Manipulation

Competent Cell Preparation

General methodolgies disclosed herein are well known to those who practice in the field of molecular biology. Additionally, there are many published sources for the general methods described herein (for example, Ausubel et al, eds., 1988) There are many different methods known in the art for preparing competent cells for transformation. One example of preparing competent cells for transformation preferably involves the following steps. E. coli strain HB101 (Boyer and Roulland-Dossoix, 1969) is streaked onto a LB plate and incubated at approximately 37° C. for approximately fifteen hours. A single colony is inoculated into 2.0 mL of LB and cultured at approximately 37° C. for approximately eight hours. The culture is then inoculated into 100.0 mL of LB and shaken vigorously until the culture reaches between 0.3 to 0.5 OD₆₀₀. The culture is chilled on ice for approximately ten minutes and the cells recovered by centrifugation at approximately 4,000 rpm for approximately ten minutes in a Sorval GS3 rotor. The pellet is then resuspended in 50.0 mL of ice-cold 0.1 M MgCl₂ and stored on ice for approximately twenty minutes. The cells are again pelleted and resuspended in 5.0 mL 0.1 M CaCl₂ and incubated on ice for approximately one hour. The suspension is mixed with 1.15 mL of 80% glycerol, and 100.0 μL aliquots are then rapidly frozen on dry ice and stored at approximately −70° C. for later use.

Purification of DNA Fragments

There are many different methods known in the art for purifying nucleic acid fragments. One example of purifying nucleic acid fragments is discussed herein in the context of the lysosomal enzyme, GC. Preferably, to isolate GC-coding DNA fragments, a restriction enzyme digested DNA or PCR sample is loaded onto an agarose gel and the fragments resolved by electrophoresis is known in the art. A gel slice containing the band representing the GC gene is cut out and sealed in 8,000 MWCO dialysis tubing with 500.0 μL ddH₂O. The tubing is placed in an electrophoresis tank and electrophoresis continued for fifteen to sixty minutes to elute the GC gene from the gel.

The solution containing the GC-encoding DNA is then collected, extracted with 500.0 μL of both phenol and chloroform: isoamyl alcohol (95:5), and precipitated with 0.25 M ammonium acetate, 2.5 volumes of 95% ethanol, and 10.0 μg yeast tRNA carrier. The nucleic acid is pelleted by centrifugation at 14,000 rpm for ten minutes, rinsed with 70% ethanol, and resuspended in 20.0 μL ddH₂O.

Ligation

There are many different methods known in the art for ligating nucleic acid fragments to one another. One example of ligating nucleic acid fragments to one another is described herein in the context of the lysosomal enzyme GC. Preferably, to ligate the GC-encoding gene into a preferred plasmid vector (discussed supra), a 20.0 μL ligation mixture is prepared that contains 50.0-200.0 ng linearized vector, a five- fold molar excess of insert DNA, 1.0 mM ATP, 50.0 MM Tris-HCl (pH 7.6), 10.0 MM MgCl₂, 1.0 mM DTT, 5% (w/v) PEG 8000, and 1 unit of T4 DNA ligase (Life Technologies). For ligation of cohesive termini, the ligation mixture is incubated at 16° C. for a length of time ranging between two to sixteen hours.

Bacterial Transformation

There are many different methods known in the art for introducing nucleic acids into bacteria. One example of transformation includes the following steps. The transformation of the lysosomal enzyme (for example, GC) expression cassette discussed herein preferably involves gently mixing 10.0 μL of ligation mixture with 100.0 μL freshly thawed competent cells, followed by incubation on ice for approximately thirty minutes. The sample is heat shocked for approximately two minutes at 42° C., then mixed with 900.0 μL of LB, and incubated at 37° C. for approximately thirty minutes. The cells are then pelleted by centrifugation at approximately 6,000 rpm, resuspended in 100.0 μL of fresh LB and spread on a LB agar plate containing 100.0 μg/mL ampicillin. The plate is then incubated overnight at approximately 37° C.

Identification of Recombinant Clones

There are many different methods known in the art for identifying transformed bacteria clones. One example of identifying recombinant clones includes the following steps. Pre-screening of individual plasmid DNAs presumed to contain a successfully ligated lysosomal enzyme gene is preferably performed using quick minipreps of several colonies. The verification of the plasmid DNAs containing the lysosomal enzyme gene is then preferably undertaken by sequencing or the restriction enzyme digestion pattern of miniprep

MiniPrep for Plasmid DNA

There are many different methods known in the art for amplifying, purifying, and identifying nucleic acids transformed into bacteria. One preferred example of amplifying, purifying, and identifying nucleic acid is discussed herein. A single colony of E. coli HB 101 transformed with a pBluescript® SK(+/−) based recombinant plasmid (Stratagene, Genbank Accession No. X52324) is inoculated into 2.0 mL LB media containing 100.0 μg/mL of ampicillin and incubated at approximately 37° C. overnight. Preferably the following day, 100.0 mL of bacterial culture is pelleted at approximately 3,000 rpm for one minute in a benchtop centrifuge. The pellet is next resuspended in 25.0 μL of ddH₂0 and vortexed vigorously with an equal volume of phenol. After centrifuging for approximately two minutes at 3,000 rpm, 15.0 μL of the supernatant is mixed with 2.5 μL of 6× DNA dye (0.25% bromophenol blue, 0.25% xylene cyanol FF and 40% (w/v) glycerol). The mixture is then analyzed on a 1.0% agarose gel for supercoiled plasmid DNA using gel electrophoresis techniques commonly known in the art. Supercoiled plasmid DNA containing the insert can be easily discriminated from plasmids without the insert because they migrate more slowly during electrophoresis than plasmids without an insert.

An alternative method for preparing mini-prep DNA is to preferably pellet 1.5 mL of an overnight bacterial culture at approximately 6,000 rpm for five minutes in the benchtop centrifuge and resuspend in 100.0 μL of Solution 1 (which preferably includes 50.0 mM glucose, 25.0 mM Tris-HCI, pH 8.0, and 10.0 mM EDTA, pH 8.0). Next, 200.0 μL of freshly prepared Solution II (which preferably includes 0.2 M NaOH and 1.0% SDS) is added and mixed gently to the suspended pellet to cause cell lysis and denature the nucleic acid. After incubating five minutes on ice, 150.0 μL of Solution III (which preferably includes 90.0 μL of 3 M potassium acetate, 17.25 μL of glacial acetic acid, and 47.25 μL ddH₂O) is added and mixed well. The resulting suspension is incubated on ice for approximately five minutes to allow the DNA to renature and the protein-nucleic acid complexes to precipitate.

After centrifuging for five minutes spin at approximately 14,000 rpm in a microcentrifuge to pellet debris, the supernatant is transferred to a fresh tube, and the aqueous phase containing the nucleic acid is extracted with 500.0 μL phenol to remove residual protein. Next the phenol is extracted using 500.0 μL of chloroform: isoamyl alcohol (95:5). Nucleic acid consisting of plasmid DNA and bacterial RNA is then precipitated with 1.0 mL of 95% ethanol and pelleted by centrifuging at approximately 14,000 rpm. The pellet is then rinsed with 70% ethanol and dissolved in 50.0 μL ddH₂O containing 20.0 μg/mL DNAse-free RNAse.

Large Scale Plasmid DNA Preparation

There are many different methods known in the art for amplifying and purifying nucleic acids from bacteria. One preferred example of amplifying and purifying nucleic acids from bacteria is discussed herein. For a large-scale preparation of the DNA plasmid, a preferred method includes the following steps. A single colony is incubated for approximately eight hours in 2.0 mL of LB containing 100.0 μg/mL ampicillin and then transferred into 250.0 mL of terrific broth, which contains 100.0 μg/mL ampicillin. The mixture is then incubated overnight. Preferably the following day, cells are pelleted by centrifugation at approximately 4,500 rpm for approximately ten minutes in a Sorval GS3 rotor. The pellet is then resuspended with 5.0 mL of Solution I (as discussed supra) and incubated for ten minutes with 1.0 mL of 10.0 mM Tris-HCl (pH 8.0) containing 100.0 μg/mL hen egg-white lysozyme. The cells are then lysed and the nucleic acid is denatured for ten minutes by adding 10.0 mL of freshly prepared Solution II, (discussed supra). The DNA is then renatured by adding 7.5 mL of Solution III (discussed supra) and then incubated on ice for approximately twenty minutes. After centrifuging at approximately 8,000 rpm in a SS23 rotor, the supernatant is mixed well with 0.6 volumes of isopropanol and then stored at room temperature for approximately ten minutes. The nucleic acid from the supernatant is precipitated by centrifuging at around 8,000 rpm for approximately ten minutes in a SS34 rotor, and is subsequently dissolved in 3.0 mL of ddH₂O.

To further purify the plasmid DNA, 3.3 g of cesium chloride and 200.0 μL of 10.0 mg/mL ethidium bromide can be added. The sample is spun at approximately 8,000 rpm in an SS34 rotor. The clear supernatant is then loaded into a 3.90 mL ultracentrifuge tube (Beckman Coulter) and centrifuged at approximately 10,000 rpm for at least five hours at 20° C. in preferably a TL-100 benchtop ultracentrifuge (Beckman Coulter) equipped with a TLN-100 rotor. After centrifugation, the band containing supercoiled plasmid DNA is recovered preferably using a 1.0 mL syringe and a 21-gauge needle. Preferably 0.5 mL of solution is collected. The ethidium bromide in the solution can be removed by extraction several times with 1.0 mL of n-butanol saturated with 4.0 mM NaCl and 10.0 mM EDTA until the solution is completely colorless.

The solution is next diluted with 3 volumes of ddH₂O, and the plasmid DNA is precipitated using 2.5 volumes of 95% ethanol. After centrifuging at approximately 10,000 rpm for around twenty minutes (preferably using a SS34 rotor), the plasmid DNA is dissolved in ddH₂O and is preferably precipitated twice using 0.25 M ammonium acetate and 2.5 volumes of 95% ethanol. The pellet is then rinsed with 70% ethanol and dissolved in ddH₂O. The DNA concentration can be determined preferably using a Beckman spectrophotometer with methods well known in the art.

Sequencing Nucleic Acids

There are many different methods known in the art for sequencing nucleic acids. One preferred example of sequencing nucleic acids is described herein. Sequencing plasmid DNA is preferably performed by PCR using fluorescent dideoxynucleotides. A 10.0 μL solution containing 1.0 μg plasmid template, 50.0 nmol of each primer, and 4.0 μL of MIX (Perkin-Elmer) is subjected to thirty PCR cycles. The PCR cycles include denaturing at 96° C. for approximately thirty seconds, annealing at 50° C. for approximately thirty seconds, and product extension at 60° C. for approximately four minutes. The product is then precipitated with 2.5 volumes of 95% ethanol, 0.25 M ammonium acetate, and 10.0 μg yeast tRNA. The dried pellet can then be analyzed using acrylamide gel electrophoresis.

Expression Vector Construction

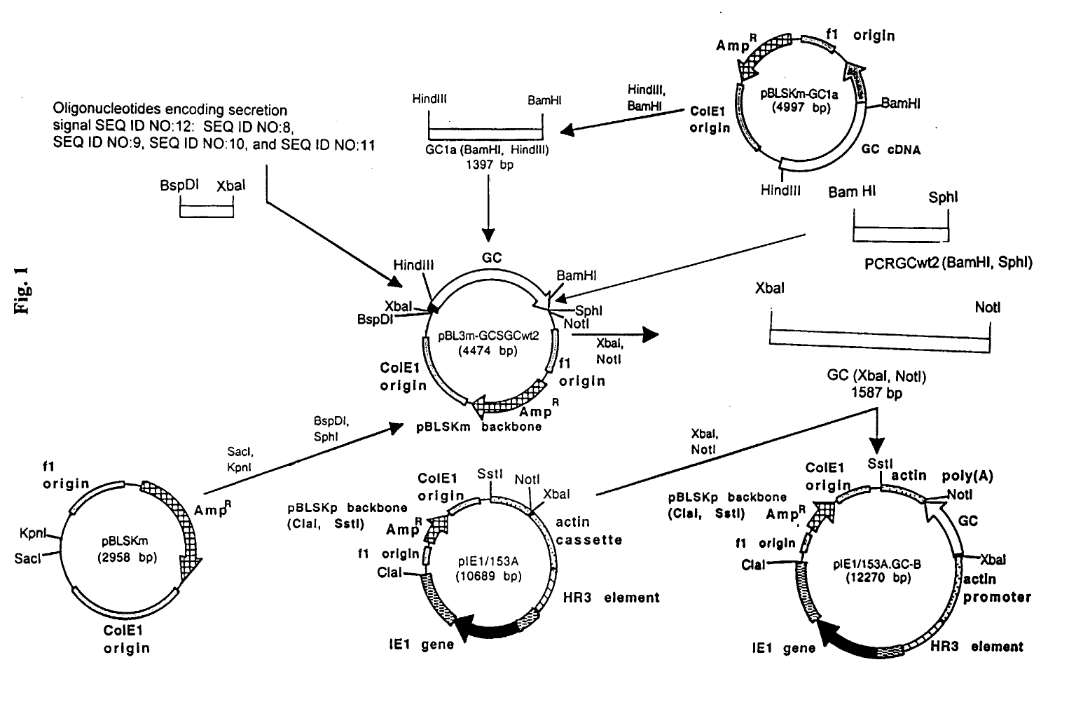

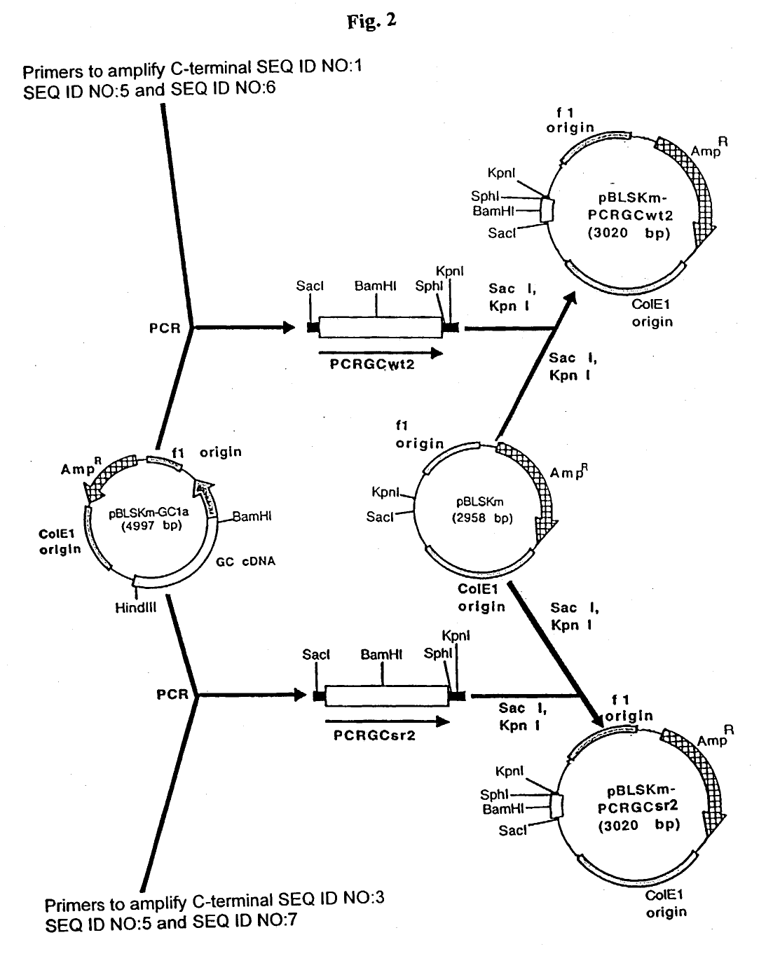

There are several different methods known in the art to construct expression vectors. However, techniques to engineer expression cassettes and transformation vectors typically include standard in vitro genetic recombination and manipulation. Vectors for the expression of human GC in insect cells were preferably constructed as shown in FIG. 1. The GC expression vector, pIE1/153A.GC-B, containing the native GC structural gene exemplified in SEQ ID NO:1 and encoding native human GC (SEQ ID NO:2) was constructed by the insertion of the 1587 bp GC expression fragment (SEQ ID NO:1) into the XbaI, NotI site of the insect expression vector pIE1/153A (described by Lu et al., 1997) to form the expression vector pIE1/153A.GC-B. Likewise, an expression vector encoding human GC with the C-terminal variant (SEQ ID NO:4; Sorge et al., 1985 and Sorge et al., 1986) was created by inserting the 1587 bp XbaI, NotI expression fragment (SEQ ID NO:3) from the pBL3m-GCSGSsr2 plasmid into the pIE1/153A expression vector to form the GC expression vector pIE1/153A.GC-C. The structural genes for human GC were constructed from three fragments of DNA generated using oligonucleotides for the N-terminal sequences (FIG. 1), PCR for the C-terminal sequences (FIG. 2), and a plasmid pBLSKm-GC1a (FIG. 1) containing human GC cDNA for the remaining sequences. Where nucleotide sequences were required for recognition and cleavage of particular restriction enzymes, oligonucleotides containing the desired sequence were annealed and ligated to specific denoted sites within the plasmid, as is well known in the art.

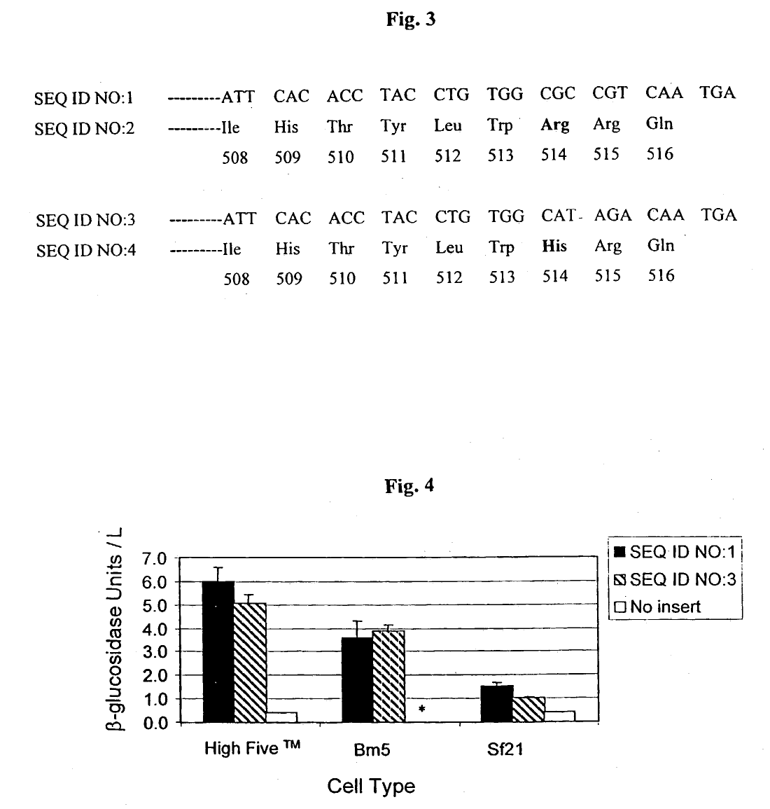

Specifically, PCR (FIG. 2) with plasmid pBLSKm-GC1a and primers SEQ ID NO:5, SEQ ID NO:6, and SEQ ID NO:7, were used to add cloning sites, remove 3′ non-coding DNA sequences, and to generate two different C-terminal amino acid coding sequences as shown in FIG. 3. SEQ ID NO: 1 has a DNA sequence altered from the native sequence but still encoding the native human GC amino acid sequence (SEQ ID NO:2). SEQ ID NO:3 encodes the C-terminal amino acid variant of human GC (Sorge et al, 1985; Sorge et al., 1986). The two sequences differ by only one amino acid. The 83 bp BamHI, SphI PCR-generated regions coding for the two different C-terminal ends of human GC were ligated to the BamHI site of the GC cDNA from the plasmid pBLSKm-GC1a (FIG. 1) in an intermediate vector with a pBluescript®SK(−) backbone (Stratagene, Genbank Accession No. X52324) and appropriate cloning sites to form constructs containing sequences coding for most of the mature GC. Finally, as shown in FIG. 1, a 96 bp BspD1, XbaI fragment constructed from four oligonucleotides, SEQ ID NO:8, SEQ ID NO:9, SEQ ID NO: 10, and SEQ ID NO:11 encoding the native GC secretion signal and the missing mature GC N-terminal amino acid sequence was ligated at the BspDI, HindIII sites of the intermediate vectors containing the partial GC coding sequences to give the final GC structural genes of SEQ ID NO:1 and SEQ ID NO:3 contained in the plasmids pBL3m-GCSGCwt2 (FIG. 1) and pBL3m-GCSGCsr2, respectively.

Generation of Transfected Insect Cells

Cell Lines

Three lepidopteran cell lines, Bm5, Sf21, and High Five™ are hosts for the expression system of the invention. However, any suitable cell line could be utilized for the expression vector disclosed herein. Bm5 cells (Dr. Iatrou, University of Calgary, Calgary, Alberta, Canada) were established from the ovarian tissue of the domesticated silkmoth Bombyx mori according to the procedure of Grace, (1967). Sf21 cells (Invitrogen) were established from the pupal ovarian tissue of the fall armyworm Spodoptera frugiperda according to Vaughn et al., (1977). BTI-TN-5B1-4 cells (commonly referred to as High Five™ cells; Invitrogen) were established from egg cell homogenates of the cabbage looper Trichoplusia ni according to Granados et al., (1994). Each of the above protocols describing the respective derivation of the Bm5 cells, Sf21 cells, and High Five™ cells are incorporated by reference herein.

Culture Media

The lepidopteran insect cells lines identified supra are routinely sub-cultured in a preferred IPL-41 insect media (Life Technologies) supplemented with 2.6 g/L tryptose phosphate broth (Difco), 0.35 g/L NaHCO₃, 0.069 mg/L ZnSO₄-7H₂O, 7.59 mg/L AIK(SO₄)₂.12H₂O and 10% fetal bovine serum (JRH Biosciences). The osmotic pressure is adjusted to 370.0 mOsm with 9.0 g/L sucrose, and pH adjusted to 6.2 with 10.0 M NaOH prior to sterile filtering through 0.2 μm filter units. For growth in serum-free media (SFM), a commercial formulation using IPL-41 media, Sf-9000 II SFM (Life Technologies), Ex-Cell™ 401 (JRH Biosciences), or ESF921 (Expression Systems LLC) can be used. Preferably, no antibiotics are used in media.

Culture Maintenance

The lepidopteran cell lines are preferably maintained in CO₂ free incubators at approximately 28° C. Cells are preferably subcultured weekly in 25 cm² T-flasks at a dilution factor of 1:5 with fresh media.

To recover frozen cell lines, one cryovial is removed from liquid nitrogen and rapidly thawed in a water bath having an approximate temperature of 28° C. The cells are then placed in a 25 cm² T-flask with 4.0 mL fresh media, and allowed to adhere for approximately five hours at approximately 28° C. The culture media containing DMSO and dead cells is then replaced with 5.0 mL fresh media.

The trypan blue exclusion method (Freshney, 1997) is preferably used to estimate the cell density and viability of cell cultures. This method is based on the fact that viable cells are impermeable to trypan blue, whereas dead cells are permeable to the dye. Typically, a cell culture sample is diluted 1:3 with 0.1% trypan blue in phosphate buffered saline (PBS; 10 mM KH₂PO₄, 2 mM NaH₂PO₄, 140 mM NaCl, 40 mM KCl), and samples counted at least twice in a hemocytometer. Although the above methods are preferred, any skilled artisan would recognize that the conditions for cell line growth, maintenance, and manipulation depend upon the cell lines used and therefore could vary within the scope of the invention.

Transfection of Insect Cells

Transfection of the cell lines identified supra with the vector identified supra could be accomplished in a variety of ways, all of which are well understood in the art. The following protocol is the preferred method for transfecting the insect cells of the expression system disclosed herein. The transfer of the expression vector comprising the expression cassette for a lysosomal enzyme into cultured insect cells is preferably performed using a cationic liposome compound commonly referred to as Lipofectin™ (Life Technologies). These positively charged liposomes are attracted to negatively charged DNA. Insect cells to be transfected are prepared by dilution in fresh media to a density of 5×10⁵ viable cells/mL, and transferring 2.0 mL of the cell suspension to each well of a six-well tissue culture plate (35.0 mm diameter, Falcon), to allow adherence overnight. A transfection solution is then prepared containing 30.0 μg/mL Lipofectin™ (Life Technologies) and 6.0 μg/mL total plasmid DNA in basal IPL-41. The lipid is initially diluted in 0.275 mL IPL-41 (Life Technologies) and incubated for forty-five minutes at room temperature. The plasmid DNA is diluted separately in 0.275 mL basal IPL-41 and then combined with the Lipofectin™ solution. The resulting solution is incubated on ice for approximately fifteen minutes. The cells are then washed twice with 1.0 mL basal IPL-41 and incubated at approximately 28° C. with 0.55 mL transfection solution per well. After approximately six hours of transfection, the cells are rinsed with basal IPL-41 followed by adding 2.0 mL complete media to the well. Approximately three days later, samples can be analyzed for transfection.

Detection and Analysis of Recombinantly Produced GC

Preparation of Total Cell Extracts for SDS-PAGE

Anyone skilled in the art will recognize that there are a variety of ways to prepare cellular extracts for SDS-PAGE. The preferred method for preparing cellular extracts for SDS-PAGE is discussed herein. Transfected insect cells are counted forty-eight to sixty hours post-transfection and pelleted at around 3,000 rpm for approximately five minutes in a microcentrifuge. The transfected cells are then washed two times with 1.0 mL of PBS. Aliquots containing 2×10⁵ viable cells are pelleted and resuspended in 40.0 μL ddH₂O and 8.0 μL 6× SDS-PAGE sample buffer. The viscosity of the samples can be reduced by mild sonication for approximately ten seconds to shear nucleic acid. Samples were boiled for 3 minutes before loading on gels.

Protein Quantiation, SDS-PAGE, Western Blot Analysis, and Amino Acid Sequencing

Detection and quantitation of recombinantly produced GC is preferably performed using protein assays, SDS-PAGE and Western blot analysis. These techniques are well-known to those skilled in the art. See for example, Coligan, et al., eds. (1989).

SDS-PAGE was performed on samples to be analyzed by amino acid sequence analysis. After transferring to PVDF membranes, proteins were stained with 0.1% Coomassie® Blue R250. Bands containing the protein of interest were excised. Amino acid sequence analysis was accomplished using an Applied Biosystems Procise 492 cLC system. Optimized standard pulsed-liquid phase cycles were used. The Coomassie® Blue stained membrane strip was cut into approximately 2.0 mm pieces and loaded into the cartridge for analysis.

Western blot analysis can be performed in different ways, as anyone skilled in the art knows. The preferred method for detecting recombinantly produced lysosomal enzyme by Western blot analysis is discussed herein in the context of GC. Sample aliquots containing recombinant proteins are resolved by electrophoresis in a SDS-containing 9% acrylamide gel (SDS-PAGE), and electroblotted onto nitrocellulose Hybond-ECL (Amersham) or Immobilon™-P membranes (Millipore Corporation) overnight at 30 V in the cold. After the transfer, the filter is blocked for one hour at room temperature in 50.0 mL PBS-0.1% Tween-20 (PBST) containing 10% (w/v) skim milk powder (PBSTM). The filter is then incubated for one hour at room temperature with 5.0 mL PBST containing GC-specific polyclonal antibody obtained from Dr. Ernst Beutler, Scripps Clinic and Research Foundation, La Jolla, Calif. and designated NN1274. The filter is then washed twice for approximately fifteen minutes with PBST, and incubated one hour with 5.0 mL PBSTM containing horseradish peroxidase-conjugated species goat anti-rabbit IgG. After washing twice with PBST, the filter is incubated with ECL chemiluminescent substrate (Amersham) according to the supplier's instructions and exposed to X-ray film. Alternatively, an ImmunoBlot Assay Kit (Bio-Rad Laboratories) containing goat anti-rabbit phosphatase, BCIP, and NBT can be used.

β- glucosidase Assay

There are a variety of protocols that could be employed to detect the activity of a recombinantly expressed lysosomal enzyme. In the context of detecting GC activity, an exemplary method to detect the GC activity is the β-glucosidase assay (adapted from Suzuki, 1978, which is incorporated herein by reference). Other enzyme assays to detect the activity of other lysosomal enzymes may be used. With respect to GC activity, the β-glucosidase assay is a widely utilized assay in Gaucher disease research and is carried out under conditions in which other, non-GC glucosidase activities are partially inhibited, i.e., by using a phosphate buffer, pH 5.9, 0.125% taurocholate, 0.15 percent Triton X-100. In this assay, the fluorometric product, 4-methylumbelliferone (4-MU) is enzymatically released from the substrate, 4-methylumbelliferyl-β-D-glucopyranoside (4MUG) by GC. Samples and controls are first serially diluted on ice in 1× Assay Buffer (40.0 mM phosphate citrate buffer pH 5.90, 0.15% Triton X-100, and 0.12% sodium taurocholate) and 20.0 μL of each sample is added to sample wells of a 96-well plate (Nunc) already containing 30.0 μL of 1× Assay Buffer. Then 50.0 μL of substrate (2 mM 4-MUG in 1× assay Buffer) is added to sample wells and after gentle shaking, the enzymatic reaction is allowed to proceed at approximately 37° C. After approximately thirty minutes to one hour, the reaction is stopped by the addition of 150.0 μL Stop Solution (0.6 M glycine pH10.7). Next, 150.0 g/L aliquots of standard containing 0 to 16,000 μM 4-MU in Stop Solution and 100.0 μL of 1× Assay Buffer are added to standard wells. The amount of fluorometric product, 4-MU is detected by measuring the 465 nm emission from wells excited at 360 nm using a HTS7000 fmax Fluorescence Microplate Reader (Molecular Devices). Linear regression of standard measurements allowed the estimation of GC activity in culture samples. One unit of GC activity (U) is defined as the amount of enzyme required to hydrolyze one micromole of 4-methylumbelliferyl-β-D-glucopyranoside (4-MUG) per minute at 37° C. Examples of this assay regarding GC expression are provided at FIG. 6 and discussed infra.

### EXAMPLE 1

### Transient Expression of Native Human GC

The expression plasmids pIE1/153A.GC-B and pIE1/153A.GC-C were generated by digesting the plasmids pBL3m-GCSGCwt2 and pBL3m-GCSGCsr2 with the restriction enzymes XbaI and NotI and inserting the nucleotide fragment encoding for the GC gene (SEQ ID NO:1 or SEQ ID NO:3) into the unique XbaI, NotI sites of the pIE1/153 A plasmid (Lu et al., 1997). This vector directs a high level of expression of heterogenous proteins through the use of the insect actin promoter, a trans-acting transcription activator, and a transcriptional enhancer. FIG. 1 illustrates the process of expression vector production. To confirm insertion and correct orientation, the 5′- and 3′- ends of the GC gene were sequenced. The different human GC genes on each of pBLSKm-PCRGCwt2 and pBLSKm-PCRGCsr2 FIG. 2) have a one amino acid difference, as exemplified in FIG. 3. The sequence encoding human GC in the BamHI, SphI pBLSKm-PCRGCwt2 fragment can be found in SEQ ID NO: 1 from nucleotide 1489 to 1560. The sequence encoding human GC in the BamHI, SphI pBLSKm-PCRGCsr2 fragment can be found in SEQ ID NO:3 from nucleotide 1489 to 1560. The amino acid at position 514 of SEQ ID NO:2 is arginine, whereas the amino acid at position 514 of SEQ ID NO:4 is histidine. Methods to construct vectors are well known in the art and exemplified supra.

Bm5 (Dr. Iatrou), Sf12 (Invitrogen), and High Five™ (Invitrogen) insect cells were transfected with the GC-encoding expression plasmid pIE1/153A.GC-B (described above), pIE1/153A.GC-C (described above), and the control plasmid pIE1/153A, which lacked the coding region for GC. The transfection protocol was described by Farrell et. al., (1998), which is incorporated herein by reference. Initial transfections were performed in media containing approximately 10% fetal bovine serum. β-glucosidase activity assays were used to quantitate the amount of GC secreted to the media three days post-transfection. The results are summarized in FIG. 4. β-glucosidase activity was observed in all three cells lines transfected with vectors encoding the native GC sequence (pIE1/153A. GC-B containing SEQ ID NO: 1) or the C-terminal variant GC sequence (pIE1/153A.GC-C containing SEQ ID NO:3). The High Five™ cells produced the greatest amount of GC activity followed by the Bm5 cells and Sf21 cells, respectively.

Three days post-transfection samples of insect cell extracts and supernatants were analyzed by Western blotting. The Western blots revealed that each cell line, independent of the GC sequence or cell type, efficiently secreted GC. After three days, approximately fivefold more GC had accumulated in the media than was present inside the transfected cells. Due to co-migration of GC with the large amount of bovine serum albumin present in the supernatant, no accurate measurement of the molecular mass of the secreted GC could be obtained. However, the insect-produced GC of either the native or variant sequence co-migrated with Cerezyme™ (Genzyme), indicating all three proteins have similar molecular masses.

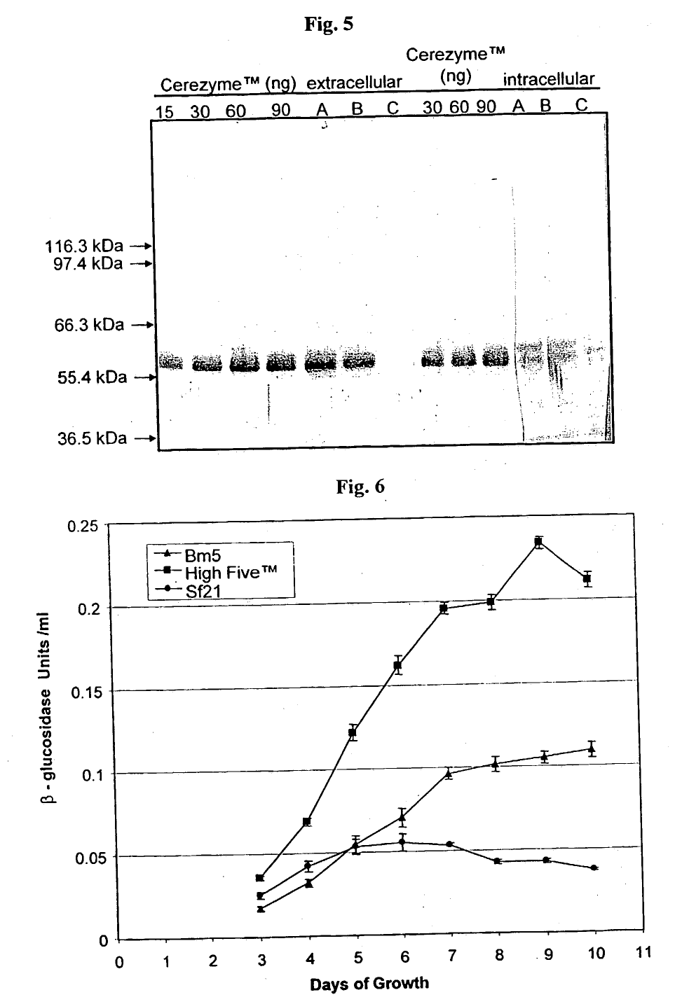

To estimate the molecular mass of the GC secreted by insect cells, High Five™, Bm5, and Sf21 cells were transfected with the GC expression plasmids pIE1/153A.GC-B and pIE1/153A.GC-C and the cell populations were maintained in EXCELL 401 serum-free media for three days. Western immunoblot analysis of media aliquots and cell extracts demonstrated that the molecular mass of the native or variant GC produced in insect cells was substantially the same as Cerezyme™ at approximately 59 kDa, and was independent of the insect cell line. GC remaining intracellularly had a molecular mass of 61.5 kDa. Insect cells in serum-free media efficiently secreted GC with five to ten-fold more GC in the media than in the cells. Results from the High Five™ cells are shown in FIG. 5. This efficiency of secretion was surprising, because human GC is not secreted extracellularly and in previous work with the baculovirus-insect cell expression system the majority of the expressed GC was found to be cell-associated ('864 patent; Grabowski, 1989). This efficient secretion of GC by insect cells is an advantage over most GC expression systems, allowing for simplified purification procedures.

### EXAMPLE 2

### GC Expression from Polyclonal Populations

To generate stably transformed insect cell lines, two antibiotic selection schemes were tested, both well established in the art:

1) Co-transfection of the expression plasmids with pBmA.HmB (Dr. Iatrou, University of Calgary, Calgary, Alberta, Canada), a selection plasmid expressing hygromycin B phosphotransferase, enables cells with successful integration events to survive in the presence of the antibiotic hygromycin B. The procedure for selecting stable cell lines using hygromycin B is discussed in Farrell et. al., (1998).

2) Co-transfection of the expression plasmids with pBmA.PAC (Dr. Iatrou, University of Calgary, Calgary, Alberta, Canada), a selection plasmid expressing puromycin acetyltransferase, enables cells with successful integration events to survive in the presence of the antibiotic puromycin.

Bm5, High Five™, and Sf21 cells were transfected in 6-well plates with a 100:1 molar ratio of expression cassette to antibiotic selection plasmid. After 48 hours recovery in non-selective conditions, the culture media was exchanged with selective media containing antibiotic. Subculturing and media exchanges were performed each week until a polyclonal population of antibiotic resistant cells was obtained and transferred to a 25 cm² T-flask. GC production by polyclonal populations decrease as faster growing, less productive clones within the population eventually dominate. Regardless of selection scheme, the polyclonal populations of Bm5, High Five m, and Sf21 cells all have the ability to express the GC protein.

Samples from each polyclonal population were taken 7 days after transformation and analyzed by a β-glucosidase assay to estimate the GC levels. The results demonstrate that the polyclonal populations of Bm5, High Five™, and Sf21 cells all have the ability to express native or C-terminal variant GC, independent of the antibiotic selection scheme. β-glucosidase levels ranged from 7-38 U/L. Activity in the negative control cells ranged from 0.4-2 U/L.

SDS-PAGE analysis and detection by Coomassie® Blue staining revealed that a band of 59 kDa is produced only in the transformed polyclonal populations. Cerezyme™ also migrated as a 59 kDa band, but had a slightly greater heterogeneity. Western immunoblot analysis confirmed that this expressed band is GC and is only present in the transformed cells. N-terminal amino acid sequence analysis of the 59 kDa band seen in the transformed insect cells confirmed its identity as GC and that insect cells are able to cleave the human GC secretion signal to produce mature GC starting at amino acid 20 (SEQ ID NO:2) as is seen for mature human GC.

### EXAMPLE 3

### GC Expression From Single Clones

Selection of clones

Co-transfection of plasmids encoding GC with plasmids encoding hygromycin B phosphotransferase or puromycin acetyltransferase created populations of GC expressing Sf21, High Five™, and Bm5 cells resistant to either puromycin and hygromycin. All populations were transfected in the presence of a 100:1 molar ratio of GC expression encoding plasmid to antibiotic resistance encoding plasmid. After culturing for two days in nonselective media, cells were selected for hygromycin or puromycin resistance.

Clones of Sf21, High Five™, and Bm5 cells expressing GC were isolated by two rounds of limited dilution cloning. In this method, cells from all populations were diluted in selective media and plated at a density of one cell/well in 96-well plates. Cells from single colony wells were reseeded and allowed to grow in selective media for 10 days, after which relative GC activity in the supernatant was determined from the β-glucosidase assay. Clones were chosen based on their high GC activity and proliferation rate and reseeded into 24-well plates. Ten days later clones were assayed for GC activity and clones with the highest GC activity and proliferation were chosen for further expansion and reseeded into 6-well plates. The most active GC expressing clones from each cell line under each selectable marker were transferred to T-flasks. GC activity was determined in culture supernatant by the β-glucosidase assay after 7 days growth. Cells at this stage were subjected to a second round of limited dilution cloning as above. The final highest producing clones were adapted to suspension cultures in serum-free media.

Production of GC

High-level production of GC by single cell clones of insect cells was demonstrated by measuring GC activity in the supernatants of suspension cultures over time. The β-glucosidase assay was employed to quantitate the activity of the GC produced by the transformed Bm5, High Five™, and Sf21 single cell clones. The β-glucosidase assay was performed as described supra. In FIG. 6, the GC activity from media was assayed daily 3-10 days after transfection. The activity level of the Bm5 produced GC peaked at day 10, whereas the activity level of the High Five™ produced GC peaked at day 9. Peak activity of the Sf21 produced GC occurred at day 6. The High Five™ clones produced the highest level of GC followed by the Bm5 and Sf21 clones. Typically, 200-300 U/L, 100-150 U/L, and 50-100 U/L were observed in the High Five™, Bm5, and Sf 21 clones, respectively. This activity was determined to be from GC, because no more than 2 U/L of endogenous glucosidase activity was seen for any untransformed cell line.

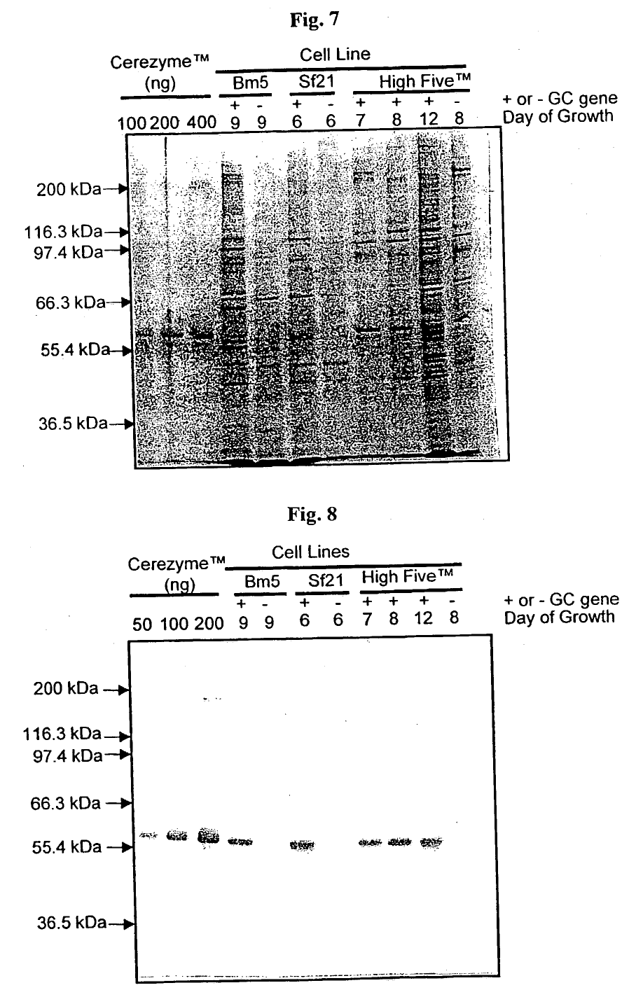

Coomassie® Blue stained SDS-PAGE gels, as exemplified in FIG. 7, demonstrated a 59 kDa band that co-migrated with Cerezyme™ and was present only in the culture supernatants of cells transformed with GC expression vectors. Typically, a secreted expression level of 25-100 mg/L was demonstrated.

Western immunoblot analysis, as exemplified in FIG. 8, using GC specific polyclonal antibody confirmed that GC is produced in all three transformed cell lines and is secreted to the media as a 59 kDa protein that co-migrates with Cerezyme™. However, Cerezyme™ is slightly more heterogeneous in size than is insect-expressed GC. The molecular mass of GC predicted by either the native (SEQ ID NO:2)or variant (SEQ ID NO:4) GC amino acid sequence is 55.6 kDa. Recombinant GC produced in CHO cells has only complex oligosaccharide chains added to only four of the five N-glycosylation sites ('892 patent). Complete remodeling of this structure would result in a protein with a maximum molecular mass of approximately 60.6 kDa. The High Five™ clones gave the highest level of GC production, followed by the Bm5, and Sf 21 clones. By comparison to the Cerezyme™ standard it was estimated that, typically, GC levels of 25-100 mg/L were produced.

Purification

Methods to purify recombinant proteins are well known to those of skill in the art. In the case of the recombinantly expressed lysosomal enzyme GC, most GC sources, such as transformed CHO cells, baculovirus-infected insect cells, or human placental tissue, require detergent for solubilization because GC is associated with the cellular fraction. In contrast, GC produced with the expression system herein described is secreted into the extracellular environment at high concentrations in a soluble form permitting a less time-consuming purification procedure to be utilized.

One non-limiting method to purify GC from the media of suspension cultures of transformed insect cells is described herein. The media containing GC is separated from cells by centrifugation and can be stored at −80° C. until further purification is desired. All chromatography steps are done at 4° C. After thawing, the media is clarified by centrifugation and filtered before applying to a Hi Prep 16/10 octyl-sepharose hydrophobic interaction column (Pharmacia) using a model 2150 high-pressure liquid chromatography system (LKB-Produkter AB). The column is equilibrated in Buffer A (50 mM Nacitrate buffer, pH 6.0; pH adjusted at room temperature). The column is washed and eluted with a linear 20 to 50% ethanol gradient in Buffer A. Eluent from the column is collected fractionally in polypropylene tubes. The fractions are analyzed for activity and purity using the glucosidase assay and SDS-PAGE, respectively. Fractions with the highest purity and activity are pooled and concentrated using YM-30 membrane concentration devices (Amicon Inc.). The concentrated pool is then further purified by high-pressure liquid chromatography using gel permeation chromatography with a 0.75×60 cm TSK G 3000SW column (Tosoh Corporation) equilibrated in Buffer A containing 40% ethanol. Eluent from the column is collected fractionally in polypropylene tubes. The fractions are analyzed for activity and purity using the β-glucosidase assay and SDS-PAGE, respectively. Fractions with the highest purity and activity are pooled and concentrated as before. The pooled concentrate is then stored at −80° C. Substantially pure enzyme is obtained.

Monosaccharide and N-glycan Analysis Regarding the Lysosomal Enzyme GC and Cerezyme™

Purified GC from a single cell clonal population of High Five™ cells and Cerezyme™ were analyzed by high pH anion-exchange chromatography coupled to pulsed amperometric detection (HPAEC-PAD) to determine the nature and quantity of sialic acid, monosaccharide, and amino-sugars. For the release and quantitation of neutral and amino-sugars, samples were hydrolyzed in 2 N HCl (4 hours, 100° C.). For the release and quantitation of sialic acids, samples were hydrolyzed in 0.1 N TFA (1 hour, 80° C.). After hydrolysis, samples were dried in a SpeedVac centrifugal evaporator, without heat. Following resuspension in water, the solutions of released monosaccharides were separated on a Dionex PA-1 anion-exchange column. Detection by pulsed amperometry was with a Dionex ED40 electrochemical detector employing a standard pulse waveform (triple potential) optimized for carbohydrate response. Standard curves for quantitation were generated from known amounts of monosaccharides. The identities of monosaccharides in the samples were assigned based on their retention time relative to standard peak retention times. GC produced in insect cells transformed with the pIE1/153A.GC-B vector contained only mannose, N-acetylglucosamine, and fucose residues, demonstrating that complex and 0-linked oligosaccharide chains do not exist on insect-produced GC. Because the GC produced by the method described herein lacks sialic acid and galactose residues, it has proportionately more terminal mannose residues, making it more bioavailable to the targeted phagocytic cells. Total monosaccharide analysis was also performed on Cerezyme™ and in contrast to the GC produced by the method described herein, was found to contain a small amount of sialic acid (0.2 mole/mole of protein), indicating incomplete remodeling.

The N-glycan structure of insect cell-expressed recombinant GC was analyzed by Endo H digestion and Western immunoblot analysis. The endoglycosidase, Endo H, specifically releases N-linked hybrid and high mannose type oligosaccharides. If a glycoprotein contains a high mannose structure, the Endo H digestion will lead to a shift in the mobility of the Endo H digested glycoprotein by SDS-PAGE analysis. When insect-produced recombinant GC samples were digested with Endo H as recommended by the manufacturer (Glyko, Inc.), and analyzed by SDS-PAGE and Western immunoblot, no difference in mobility between undigested and Endo H treated GC samples was observed. This demonstrates that the major portion of GC produced by the method described herein does not contain the high mannose type of N-glycan. Proteins with the high mannose type of N-glycan have proportionately fewer terminating mannose residues than proteins without the high mannose type of N-glycan. Because the terminal mannose residues mediate bioavailability in the cells that accumulate glucocerebroside, the lack of these chains in GC produced by the method described herein makes it a more effective product than GC isolated from human placenta (the '838 patent) and from baculovirus production systems (the '864 patent; Martin et al., 1988).

Terminal mannose residues in the N-glycans of GC produced by the method herein were detected by digestion with exoglycosidases α- mannosidase II and α-mannosidase VI and Western blot analysis. α-mannosidase II (MANase II,Glyko, Inc.) has a broad specificity, cleaving Manα1-2,3 and 6 linkages, but does not cleave a single Manα1-6 linked to the core β-mannose residue. α-mannosidase VI (MANase VI, Glyko, Inc.) specifically cleaves unbranched Manα1-6 linked to the core -mannose residue. The amount of α-mannose that can be removed from a glycoprotein by sequential digestion with α-annosidase II and α-mannosidase VI is directly related to the amount of terminal mannose in the N-glycans. The amount of α-mannose in recombinant GC samples was determined by Western blotting using a biotinylated Hippeastrum Hybrid Lectin (Vector Laboratories) that specifically binds to α-mannose residues according to the manufacturer's enclosed protocol. Specifically, purified GC samples at 0.45 mg /ml were digested as recommended by the manufacturer (Glyko, Inc.) with mannosidase II at a final concentration of 50 Units/ml for 19 hrs at 37° C. Next, half of the mannosidase II-digested GC sample was further digested with mannosidase VI as recommended by the manufacturer (Glyko, Inc.) at a final concentration of 20 Units/ml for 23 hrs at 37° C. 60 ng of single or double mannosidase digested GC samples, undigested GC samples, and Cerezyme™ were analyzed by Western blotting. The results demonstrated that greater than 95% of α-mannose residues were removed from recombinant insect-expressed GC by the sequential digestion with α-mannosidase II and α-mannosidase VI indicating that almost all the α-mannose residues of recombinant insect GC as it is directly produced are contained in N-glycan chains that have a terminal mannose residue and are not located in chains that terminate in other sugars, i.e. sialic acid, galactose, or N-acetylglucosamine residues. Duplicate blots analyzed using GC specific polyclonal antibody confirmed that the decrease in the lectin binding to the mannosidase digested samples was not due to a loss of GC.

The α-mannose residues present in GC produced by the method disclosed herein are not contained in high mannose type N-glycans and are not blocked by N-acetylglucosamine residues, sialic acid residues, nor galactose residues. A high proportion of terminal mannose residues is thought to be critical for uptake of GC into the non-parenchymal cells that accumulate glucocerebroside, such as macrophages or Kupffer cells. The high proportion of terminal α-mannose residues of the recombinant GC produced directly by the transformed insect cells makes this production system particularly useful and efficient in comparison to systems that require enzymatic remodeling of GC by the sequential digestion of three glycosidases to expose terminal mannose residues.

Patents

U.S. Pat. No. 5,236,838 Rasmussen, et al.

U.S. Pat. No. 5,549,892 Friedman, et al.

U.S. Pat. No. 5,759,809 Iatrou, et al.

U.S. Pat. No. 5,929,304 Radin, et al.

U.S. Pat. No. 6,074,861 Ginns, et al.

Other Publications

Altmann (1997) “More than silk and honey—or, can insect cells serve in the production of therapeutic glycoproteins?” Glycoconj.J. 14, 643-646.

Ashwell et al. (1974) “The role of surface carbohydrates in the hepatic recognition and transport of circulating glycoproteins.” Adv.Enzymol.Relat.Areas.Mol.Biol. 41, 99-128.

Ausubel et al. eds., (1988) Current Protocols in Mol. Biol, Vol. I, Chapters 1-3; Vol. III, Chapters 15 and 16, Greene Pub. Associates; Wiley-Interscience, New York.

Bakker et al. (2001) “Galactose-extended glycans of antibodies produced by transgenic plants.” Proc.Natl.Acad.Sci.U.S.A. 98, 2899-2904.

Baynes et al. (1976) “Effect of glycosylation on the in vivo circulating half-life of ribonuclease.” J Biol.Chem. 251, 6016-6024.

Boyer et al. (1969) “A complementation analysis of the restriction and modification of DNA in Escherichia coli.” J Mol.Biol. 41, 459-472.

Coligan et al. eds., (1995) Current Protocols in Protein Science, Vol. I, Chapters 3 and 10, Greene Pub. Associates; Wiley-Interscience: New York.

Doran et al. (2000) “Foreign protein production in plant tissue cultures.” Curr.Opin.Biotechnol 11, 199-204.

Farrell et al. (1998) “High-level expression of secreted glycoproteins in transformed lepidopteran insect cells using a novel expression vector” Biotechnol.Bioeng. 60, 656-663.

Freshney et al. eds., (1994) Culture of Animal Cells: A Manual of Basic Technique, pp288-289, Wiley-Liss, Inc., New York.

Furbish et al. (1981) “Uptake and distribution of placental glucocerebrosidase in rat hepatic cells and effects of sequential deglycosylation” Biochim.Biophys.Acta 673, 425-434.

Grabowski et al. (1989) “Expression of functional human acid beta-glucosidase in COS-1 and Spodoptera frugiperda cells.” Enzyme 41, 131-142.

Grace (1967) “Establishment of a line of cells from the silkworm Bombyx mori.” Nature 216, 613.

Granados et al. (1994) “A new insect cell line from Trichoplusia ni (BTI-Tn-5B1-4) susceptible to Trichoplusia ni single enveloped nuclear polyhedrosis virus.” J.Invertebr.Pathol. 64, 260-266.

Hollister et al. (2001) “Engineering lepidopteran insect cells for sialoglycoprotein production by genetic transformation with mammalian beta 1,4-galactosyltransferase and alpha2,6-sialyltransferase genes.” Glycobiology 11, 1-9.

Huybrechts et al. (1992) “Nucleotide sequence of a transactivating Bombyx mori nuclear polyhedrosis virus immediate early gene.” Biochim.Biophys.Acta 1129, 328-330.

Kawasaki et al. (1978) “Isolation and characterization of a mannan-binding protein from rabbit liver.” Biochem.Biophys Res.Commun. 81, 1018-1024.

Kubelka et al. (1994) “Structures of the N-linked oligosaccharides of the membrane glycoproteins from three lepidopteran cell lines (Sf-21, IZD-Mb-0503, Bm-N).” Arch.Biochem.Biophys. 308, 148-157.

Kulakosky et al. (1998) “N-glycosylation of a baculovirus-expressed recombinant glycoprotein in three insect cell lines.” In Vitro Cell Dev.Biol.Anim. 34, 101-108.

Lennon et al. (1996) “The I.M.A.G.E. Consortium: an integrated molecular analysis of genomes and their expression.” Genomics 33, 151-152.

Lu et al. (1997) “A baculovirus (Bombyx mori nuclear polyhedrosis virus) repeat element functions as a powerful constitutive enhancer in transfected insect cells” J.Biol. Chem. 272, 30724-30728.

Martin et al. (1988) “Glycosylation and processing of high levels of active human glucocerebrosidase in invertebrate cells using a baculovirus expression vector.” DNA 7, 99-106.

Noronkoski et al. (1997) “Influence of L-fucose attached alpha 1—>6 to the asparagine-linked N-acetylglucosamine on the hydrolysis of the N-glycosidic linkage by human glycosylasparaginase.” Glycobiology 7, 217-220.

Pastores et al. (1993) “Enzyme therapy in Gaucher disease type 1: dosage efficacy and adverse effects in 33 patients treated for 6 to 24 months” Blood 82, 408-416.

Sorge et al. (1985) “Molecular cloning and nucleotide sequence of human glucocerebrosidase cDNA” Proc.Natl.Acad.Sci.U.S.A. 82, 7289-7293.

Sorge et al. (1986) “Molecular cloning and nucleotide sequence of human glucocerebrosidase cDNA Correction” Proc.Natl.Acad.Sci.U.S.A. 83, 3567-3567.

Stahl et al. (1978) “Evidence for receptor-mediated binding of glycoproteins, glycoconjugates, and lysosomal glycosidases by alveolar macrophages.” Proc.Natl.Acad.Sci.U.SA. 75, 1399-1403.

Staudacher et al. (1999) “Fucose in N-glycans: from plant to man.” Biochim Biophys Acta 1473, 216-236.

Takahashi et al. (1999) “N-glycan structures of murine hippocampus serine protease, neuropsin, produced in Trichoplusia ni cells.” Glycoconj.J 16, 405-414.

Tsuji et al. (1986) “Nucleotide sequence of cDNA containing the complete coding sequence for human lysosomal glucocerebrosidase” J.Biol.Chem. 261, 50-53.

van Ree et al. (2000) “Beta(1,2)-xylose and alpha(1,3)-fucose residues have a strong contribution in IgE binding to plant glycoallergens.” J Biol.Chem. 275, 11451-11458.

Vaughn et al. (1977) “The establishment of two cell lines from the insect Spodoptera frugiperda (Lepidoptera; Noctuidae).” In Vitro 13, 213-217.

We claim:

1. A pharmaceutical composition comprising clinically effective recombinant glucocerebrosidase synthesized with an expression system comprised of an insect cell transformed with a vector encoding glucocerebrosidase, wherein the expression system synthesizes clinically effective glucocerebrosidase.

2. The pharmaceutical composition of claim 1, wherein the vector encoding glucocerebrosidase contains SEQ ID NO:1.

3. The pharmaceutical composition of claim 1, wherein the vector encoding glucocerebrosidase contains SEQ ID NO:3.

4. The pharmaceutical composition of claim 1, wherein the vector encoding glucocerebrosidase contains a secretion signal of amino acids 1-19 in SEQ ID NO:12.

5. The pharmaceutical composition of claim 1, wherein the vector encoding glucocerebrosidase also contains genetic elements functionally linked to glucocerebrosidase to enhance the expression of glucocerebrosidase.

6. The pharmaceutical composition of claim 5, wherein a genetic element that enhances the expression of glucocerebrosidase is the actin gene promoter from the genome of Bombyx mori.

7. The pharmaceutical composition of claim 5, wherein a genetic element that enhances the expression of glucocerebrosidase is the 1.2 kB enhancer from the genome of Bombyx mori NPV.

8. The pharmaceutical composition of claim 5, wherein a genetic element that enhances the expression of glucocerebrosidase is the IE-1 gene from the genome of Bombyx mori.

9. The pharmaceutical composition of claim 1, wherein the insect cell is a Bombyx mori insect cell.

10. The pharmaceutical composition of claim 1, wherein the insect cell is a Spodoptera frugiperda insect cell.

11. The pharmaceutical composition of claim 1, wherein the insect cell is a Trichoplusia ni insect cell.

12. The pharmaceutical composition of claim 1, wherein the produced glucocerebrosidase possesses asparagine-linked terminal mannose residues.

13. A method for treating individuals with deficiencies in glucocerebrosidase, wherein the method includes introducing into individuals with deficiencies in glucocerebrosidase recombinantly produced glucocerebrosidase from insect cells, wherein the recombinantly produced glucocerebrosidase is produced by insect cells in a clinically effective form.

14. An expression system comprised of an insect cell transformed with a vector encoding glucocerebrosidase that synthesizes clinically effective glucocerebrosidase.

15. The expression system of claim 14, wherein the vector encoding glucocerebrosidase contains SEQ ID NO:1 .

16. The expression system of claim 14, wherein the vector encoding glucocerebrosidase contains SEQ ID NO:3.

17. The expression system of claim 14, wherein the vector encoding glucocerebrosidase contains a secretion signal of amino acids 1-19 in SEQ ID NO:12.

18. The expression system of claim 14, wherein the vector encoding glucocerebrosidase also contains genetic elements functionally linked to glucocerebrosidase to enhance the expression of glucocerebrosidase.

19. The expression system of claim 18, wherein a genetic element that enhances the expression of glucocerebrosidase is the actin gene promoter from the genome of Bombyx mori.

20. The expression system of claim 18, wherein a genetic element that enhances the expression of glucocerebrosidase is the 1.2 kB enhancer from the genome of Bombyx mori NPV.

21. The expression system of claim 18, wherein a genetic element that enhances the expression of glucocerebrosidase is the IE-1 gene from the genome of Bombyx mori.

22. The expression system of claim 14, wherein the insect cell is a Bombyx mori insect cell.

23. The expression system of claim 14, wherein the insect cell is a Spodoptera frugiperda insect cell.

24. The expression system of claim 14, wherein the insect cell is a Trichoplusia ni insect cell.

25. The expression system of claim 14, wherein the produced glucocerebrosidase possesses asparagine-linked terminal mannose residues.

26. A method of producing clinically effective recombinant glucocerebrosidase comprising the steps of:
(a) developing a vector that encodes a gene for glucocerebrosidase;
(b) introducing the vector into at least one insect cell that is capable of receiving the vector and serving as host to the vector;
(c) nurturing the at least one insect cell comprising the vector so that the glucocerebrosidase gene of the vector is successfully transcribed and translated into glucocerebrosidase in its clinically effective form; and
(d) harvesting the glucocerebrosidase.

27. The method of claim 26, wherein the vector additionally encodes an enhancer functionally linked to glucocerebrosidase.

28. The method of claim 26, wherein the vector additionally encodes a promoter functionally linked to glucocerebrosidase.

29. The method of claim 26, wherein the vector additionally encodes a structural gene that increases transcription.

30. The method of claim 26, wherein the vector additionally encodes a secretion signal.

31. The method of claim 26, wherein the glucocerebrosidase gene is SEQ ID NO:1.

32. The method of claim 26, wherein the glucocerebrosidase gene is SEQ ID NO:3.

33. The method of claim 27, wherein the enhancer is 1.2 kB from the Bombyx mori NPV genome.

34. The method of claim 28, wherein the promoter is the actin promoter from the genome of Bombyx mori.

35. The method of claim 29, wherein the structural gene that increases transcription is the IE-1 gene of the Bombyx mori genome.

36. The method of claim 30, wherein the signal sequence causing glucocerebrosidase secretion is amino acids 1-19 of SEQ ID NO:12.

37. The method of claim 26, wherein the insect cell is a Bombyx mori insect cell.

38. The method of claim 26, wherein the insect cell is a Spodoptera frugiperda insect cell.

39. The method of claim 26, wherein the insect cell is a Trichoplusia ni insect cell.

40. The method of claim 26, wherein the produced glucocerebrosidase possesses asparagine-linked terminal mannose residues.

41. A method of producing clinically effective recombinant glucocerebrosidase comprising the steps of:
(a) creating a vector encoding a gene for glucocerebrosidase with a signal sequence for secretion functionally linked to an enhancer and a promoter, wherein the vector also encodes a structural gene that increases transcription as well as a structural gene that is a detectable marker;
(b) introducing the vector into an insect cell;
(c) growing the transformed insect cell under conditions so that it can optimally synthesize and secrete clinically effective glucocerebrosidase and create a multitude of cells that can synthesize and secrete clinically effective glucocerebrosidase; and
(d) collecting recombinantly synthesized clinically effective glucocerebrosidase from the extracellular environment.

42. The method of claim 41, wherein the glucocerebrosidase gene is SEQ ID NO:1.

43. The method of claim 41, wherein the glucocerebrosidase -gene is SEQ ID NO:3

44. The method of claim 41, wherein the signal sequence causing glucocerebrosidase secretion is amino acids 1-19 of SEQ ID NO:12.

45. The method of claim 41, wherein the promoter is the actin promoter from the genome of Bombyx mori.

46. The method of claim 41, wherein the enhancer is 1.2 kB from the Bombyx mori NPV genome.

47. The method of claim 41, wherein the structural gene that increases transcription is the IE-1 gene of the Bombyx mori genome.

48. The method of claim 41, wherein the structural gene that is a detectable marker confers ampicillin resistance.

49. The method of claim 41, wherein the insect cell is a Bombyx mori insect cell.

51. The method of claim 41, wherein the insect cell is a Spodoptera frugiperda insect cell.

52. The method of claim 41, wherein the insect cell is a Trichoplusia ni insect cell.

53. The method of claim 41, wherein the produced glucocerebrosidase possesses asparagine-linked terminal mannose residues.


SEQUENCE LISTING (12 sequences)

SEQ ID NO: 1 - DNA, 1592 bp, Homo sapiens
  CDS (13)..(1563)
ctagaattcaaaatggctggctcgttaacaggattgcttctacttcaggcagtgtcgtgggcatccggagctagaccttgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgccgtcaatgagacgcatgcggtacccgaattcagcggcc

SEQ ID NO: 2 - protein, 516 aa, Homo sapiens
MAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 3 - DNA, 1593 bp, Homo sapiens
  CDS (13)..(1563)
ctagaattcaaaatggctggcagcctcacaggtttgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcatagacaatgagacgcatgctggtacccgaattcagcggcc

SEQ ID NO: 4 - protein, 516 aa, Homo sapiens
MAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 5 - DNA, 36 bp, Homo sapiens
  misc_feature (1)..(36)
ccctgagctctgtgcaccccgatggctctgctgttg

SEQ ID NO: 6 - DNA, 48 bp, Homo sapiens
  misc_feature (1)..(48)
cgtggtaccggcatgcgtctcattgacggcgccacaggtaggtgtgaa

SEQ ID NO: 7 - DNA, 49 bp, Homo sapiens
  misc_feature (1)..(49)
cgaggtaccagcatgcgtctcattgtctatgccacaggtaggtgtgaat

SEQ ID NO: 8 - DNA, 41 bp, Homo sapiens
  misc_feature (1)..(41)
cgatgtctagaattcaaaatggctggctcgttaacaggatt

SEQ ID NO: 9 - DNA, 55 bp, Homo sapiens
  misc_feature (1)..(55)
gcttctacttcaggcagtgtcgtgggcatccggagctagaccttgcatccctaaa

SEQ ID NO: 10 - DNA, 44 bp, Homo sapiens
  misc_feature (1)..(44)
agcttttagggatgcaaggtctagctccggatgcccacgacact

SEQ ID NO: 11 - DNA, 54 bp, Homo sapiens
  misc_feature (1)..(54)
gcctgaagtagaagcaatcctgttaacgagccagccattttgaattctagacat

SEQ ID NO: 12 - protein, 27 aa, Homo sapiens
  SIGNAL (1)..(27)
MAGSLTGLLLLQAVSWASGARPCIPKS